(19) United States
(12) Patent Application Publication
(10) Pub. No.: US 2026/0314980 A1
(43) Pub. Date: Oct. 15, 2026

(54) Systems and Methods for Distributed Electroencephalography Synchronization

(71) Applicant: Dimitrios Kouzis-Loukas, Brooklyn, NY (US)

(72) Inventor: Dimitrios Kouzis-Loukas, Brooklyn, NY (US)

(21) Appl. No.: 19/640,310

(22) Filed: Apr. 6, 2026

Related U.S. Application Data
(60) Provisional application No. 63/786,843, filed on Apr. 10, 2025.

(51) Int. Cl. A61B 5/31; H03L 7/07; H03L 7/087; H03L 7/099

(52) U.S. Cl. CPC A61B 5/31; H03L 7/07; H03L 7/087; H03L 7/0991

(57) ABSTRACT

Systems and methods for distributed electroencephalography in accordance with embodiments of the invention are illustrated. One embodiment includes a sensor platform for a distributed sensor system, comprising a receiver, a phase locked loop (PLL) device, comprising a first phase detector configured to receive a reference signal from the receiver, a first low pass filter configured to receive an output of the first phase detector, and a first voltage controlled oscillator configured to receive an output of the first low pass filter, and an analog-to-digital converter (ADC) configured to produce a digital output signal representing a plurality of samples of an input analog signal taken over a conversion cycle, where the conversion cycle timing is controlled by an output clock signal produced by the PLL device, where the first phase detector is further configured to receive a conversion cycle signal produced by the ADC when a given conversion cycle ends.

### CROSS-REFERENCE TO RELATED APPLICATIONS

The current application claims the benefit of and priority under 35 U.S.C. §119(e) to U.S. Provisional Patent Application No. 63/786,843 entitled “Systems and Methods for Distributed Electroencephalography Synchronization” filed Apr. 10, 2025. The disclosure of U.S. Provisional Patent Application No. 63/786,843 is hereby incorporated by reference in its entirety for all purposes.

### FIELD OF THE INVENTION

The present invention generally relates to clock synchronization circuitries for distributed devices, namely synchronization for electroencephalography signal collection by synchronizing analog-to-digital converter conversion cycles.

### BACKGROUND

Electroencephalography (EEG) is a non-invasive neural recording method in which electrodes placed on a user's scalp in order to measure brain activity. EEG signals are recorded as amplitude over time and can be used for many different applications such as patient monitoring, attention measurement, brain-computer interfacing, and many more.

Analog-to-digital converters (ADCs) are electronic devices which convert continuous-time and continuous-amplitude analog signals into discrete-time and discrete amplitude digital signals. There are many different ADC architectures with various tradeoffs. Delta-sigma ADCs, for example, typically use a 1-bit digital-to-analog (DAC) converter, filtering, and oversampling to achieve highly accurate, high-resolution conversions. However, due to the oversampling requirements, delta-sigma ADCs require faster clocks than other ADCs. Delta-sigma ADCs also use more complex digital filters compared to other ADCs.

A conversion cycle is a discreet ADC converter operation referring to the process of converting the input signal into a digital result. When performed by a successive-approximation register (SAR) ADC, for example, the conversion occurs after the sample is acquired. For delta-sigma ADCs, a conversion cycle refers to the period between each data output. With delta-sigma ADCs, each digital output is based on modulator results from several time periods.

Clock drift is a phenomenon where a clock does not run at the exact same rate as a reference clock. All clocks are subject to drift, requiring some form of synchronization to keep them from diverging. A phase locked loop (PLL) is a system that generates an output signal whose phase is fixed relative to the phase of an input signal. PLLs have commonly been used for clock synchronization, among other signal processing applications.

### SUMMARY OF THE INVENTION

Systems and methods for distributed electroencephalography in accordance with embodiments of the invention are illustrated. One embodiment includes a sensor platform for a distributed sensor system, comprising a receiver, a phase locked loop (PLL) device, comprising a first phase detector configured to receive a reference signal from the receiver, a first low pass filter configured to receive an output of the first phase detector, and a first voltage controlled oscillator configured to receive an output of the first low pass filter, and an analog-to-digital converter (ADC) configured to produce a digital output signal representing a plurality of samples of an input analog signal taken over a conversion cycle, where the conversion cycle timing is controlled by an output clock signal produced by the PLL device, where the first phase detector is further configured to receive a conversion cycle signal produced by the ADC when a given conversion cycle ends.

In a further embodiment, the reference signal is a 1 pulse per second signal, and the first low pass filter cutoff frequency is between 40 mHz and 60 mHz.

In still another embodiment, the phase locked loop device further comprises a second phase detector configured to receive a first clock signal from the first voltage controlled oscillator and a second clock signal from a second voltage controlled oscillator, a second low pass filter configured to receive an output of the second phase detector, and the second voltage controlled oscillator configured to receive an output of the second low pass filter, and output a second clock signal, wherein the second clock signal is the output clock signal.

In a still further embodiment, the reference signal is a 1 pulse per second signal, and the second low pass filter has a cutoff frequency between 90 kHz and 110 kHz.

In yet another embodiment, the first phase detector, the first low pass filter, and the first voltage controlled oscillator includes a digital PLL.

In a yet further embodiment, the second phase detector, the second low pass filter, and the second voltage controlled oscillator includes an analog PLL.

In another additional embodiment, the method further includes steps for a memory configured to store the digital output signal.

In a further additional embodiment, the ADC is a delta sigma ADC.

In another embodiment again, the input analog signal is an electroencephalography signal.

In a further embodiment again, the reference signal is a global positioning system signal.

In still yet another embodiment, the reference signal is an n-pulse per second signal.

In another embodiment, the reference signal is additionally used to timestamp the digital output signal.

One embodiment includes a distributed sensor system, comprising a plurality of distributed sensor platforms, each comprising a receiver, a phase locked loop (PLL) device, comprising a first phase detector configured to receive a reference signal from the receiver, a first low pass filter configured to receive an output of the first phase detector, and a first voltage controlled oscillator configured to receive an output of the first low pass filter, and an analog-to-digital converter (ADC) configured to produce a digital output signal representing a plurality of samples of an input analog signal taken over a conversion cycle, where the conversion cycle timing is controlled by an output clock signal produced by the PLL device, where the first phase detector is further configured to receive a conversion cycle signal produced by the ADC when a given conversion cycle ends.

In still yet another additional embodiment, each distributed sensor platform in the plurality of distributed sensor platforms further comprises a second phase detector configured to receive a first clock signal from the first voltage controlled oscillator and a second clock signal from a second voltage controlled oscillator, a second low pass filter configured to receive an output of the second phase detector, and the second voltage controlled oscillator configured to receive an output of the second low pass filter, and output a second clock signal, wherein the second clock signal is the output clock signal.

In a still yet further embodiment, each ADC is a delta sigma ADC.

In still another additional embodiment, the input analog signal is an electroencephalography signal.

In a still further additional embodiment, the reference signal is an n-pulse per second signal.

In a further additional embodiment again, the reference signal is additionally used to timestamp the digital output signal.

In still another embodiment again, the method further includes steps for a server configured to receive digital output signals from each distributed sensor platform.

In a still further embodiment again, the method further includes steps for a reference signal generator.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

FIG. 1 is a system architecture for a synchronized distributed sensor system in accordance with an embodiment of the invention.

FIG. 2 is a block diagram for a sensor platform in accordance with an embodiment of the invention.

FIG. 3 is another block diagram for a sensor platform in accordance with an embodiment of the invention.

FIG. 4 is a flow chart for a synchronization process for a synchronized distributed sensor platform in accordance with an embodiment of the invention.

The description and claims will be more fully understood with reference to the following figures and data graphs, which are presented as exemplary embodiments of the invention and should not be construed as a complete recitation of the scope of the invention.

### DETAILED DESCRIPTION

Synchronization of distributed devices is a longstanding problem with many different solutions providing various results. As the degree of required synchronization increases, synchronization solutions tend to become more complex and expensive. These solutions become even more expensive when the devices to be synchronized are distributed across the globe. For real-time data collection across a distributed sensor network, synchronization can often be a matter of project feasibility. Sensors that produce continuous analog signals that must be sampled with extreme accuracy are common when comparing signals from two sources at the exact same time.

An example of this discussed herein is electroencephalogram (EEG) synchronization. EEG measures brain activity using electrodes that record continuous analog signals. These analog signals are typically converted to digital signals for processing. High accuracy is desirable for many EEG applications for many reasons including (but not limited to) high information density in neural activity, and critical output applications like brain-computer interfacing and patient monitoring. For many research and practical applications, it is useful to collect EEG measurements across a number of users at the same time for comparison of neural activity in response to the same stimuli or remote collaboration activity. This problem is difficult to solve when users are in the same room, let alone distributed across the globe. While EEG is discussed in the below, as can be readily appreciated, synchronization of any arbitrary systems can be achieved using similar methods and hardware/

Systems and methods described herein solve this problem using specialized synchronization circuitries. In numerous embodiments, delta-sigma analog-to-digital converters are used to record EEG signals because they provide high accuracy measurements. However, delta-sigma ADCs typically have large penalties on restarting, and thus typically use a continuous recording mode for sampling. Delta-sigma ADCs do not have a “single” sampling time, but rather sample over an interval spanning multiple clock cycles called a “conversion cycle.” Therefore, there is no single point in time where each sample was taken. The sample instead represents the value of the signal over the entire conversion cycle. For example, a delta-sigma ADC may have a 2 MHz clock taking 1 million samples per second, but the output of the delta-sigma process delivers 16,000 samples per second.

Due to inherent clock drift, two delta-sigma ADCs started at the same time will eventually diverge in their conversion cycle timing. This raises a critical issue with delta-sigma ADCs: they have large time penalties on restarting, and thus typically use a continuous mode for sampling. Turning off the delta-sigma ADCs periodically and synchronously restarting them is a lower effort, lower complexity solution, but results in periods where no measurements can occur. An additional solution is to use a master clock signal input into all ADCs. However, master clock signals typically are provided to all units via a wire to reduce drift and error, which is difficult or impossible in distributed scenarios.

To improve on this, systems and methods described herein synchronize the conversion cycles of delta-sigma ADCs using a reference signal and a phase locked loop (PLL) to adjust the ADC external clock frequency to continuously realign the conversion cycle window across distributed devices. This ensures that the output samples of the delta-sigma ADC correspond to the same time interval. Unlike traditional master clock systems or disciplined oscillators, the synchronization signal provided in embodiments of the invention is not a conventional clock signal intended to maintain a globally consistent frequency or phase. Instead, the provided signal is a modulated synchronization signal that is input to the clock input of a sensor platform. This synchronization signal ensures that internal processes within the sensor platform remain temporally aligned with a reference clock, such as a global positioning system (GPS) signal, without requiring the synchronization signal itself to maintain a fixed frequency or phase consistency across multiple locations.

In many embodiments, the reference signal is a 1-pulse-per-second (1 pps) signal such as (but not limited to) a global positioning system (GPS) signal. GPS signals are useful as they are already highly synchronized and available globally. In some embodiments, additional hardware can be used to bring a GPS signal inside a building to account for signal degradation when passing through walls. In numerous embodiments, the reference signal can be locally generated for local distributed systems. Different reference signals can be used in different locations, and additional circuitry can be used to synchronize the generation of the different reference signals. For example, n-pulse-per-second signals can be used, and are not restricted to a single pulse per second. Indeed, any regular signal can be used as appropriate to the requirements of specific applications of embodiments of the invention.

Configuration of the PLL is an additional challenge. In many embodiments, the PLL is configured to achieve sub-microsecond level synchronization of the conversion cycle of the ADC. In various embodiments, this requires clock stability sufficient to lose less than approximately 1/2,048,000 seconds every second. If the reference signal is lost (e.g. clouds cause loss of GPS), the PLL can continue to run. When the reference signal returns, it is used to tune the ADC external clock frequency to realign the conversion cycle to the reference signal. In numerous embodiments, to achieve both sub-microsecond synchronization and tuning capabilities, the PLL includes an analog PLL (APLL) and a digital PLL (DPLL) in a cascade topology. In some embodiments, the DPLL provides a very low bandwidth PLL filter, e.g. 50 mHz.

It is further contemplated that other applications outside the scope of EEG collection can benefit from synchronized delta-sigma ADCs. Additionally, in many embodiments, the ADCs are not required to be delta-sigma ADCs and can be any ADC architecture that produces output samples aggregating multiple subsamples over a conversion cycle. Therefore, it is contemplated that when discussing embodiments of the invention below, ADC refers to ADCs that produce output samples aggregating multiple subsamples over a conversion cycle such as, but not limited to, delta-sigma ADCs.

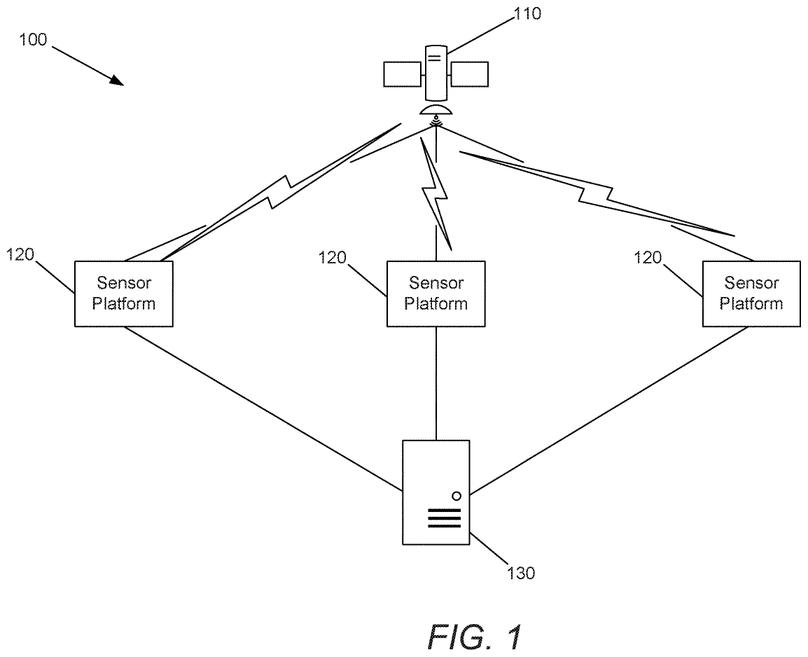

A distributed synchronized sensor network in accordance with an embodiment of the invention is illustrated in FIG. 1. System 100 includes a reference signal generator 110. In numerous embodiments, the reference signal generator is a GPS satellite. When GPS satellites are used to generate the reference signal, multiple satellites (not pictured) can be used, which are all synchronized to each other. However, in various embodiments, terrestrial reference signal generators can be used such as (but not limited to) radio towers, antennas, or any other broadcasting system capable of generating and broadcasting a reliable reference signal. In many embodiments, reference signal generator produces an n-pulse per second signal with high accuracy. For example, GPS satellites generate extremely reliable 1 pps signals.

System 100 further includes a number of different sensor platforms 120. While FIG. 1 illustrates 3 sensor platforms, any number of sensor platforms can be used as appropriate to the requirements of specific applications of embodiments of the invention. In many embodiments, the sensor platforms are EEG recording devices. Sensor platforms include ADCs and synchronization circuitries that adjust the conversion cycle of the ADC based on the received reference signal. The sensor platforms transmit their recorded data to server 130. In numerous embodiments, the sensor platforms communicate with servers via a network such as (but not limited to) the Internet. In some embodiments, the sensor platforms communicate with the server via a local network. In any case, networks that connect the sensor platforms and the server can be wired networks, wireless networks, or any combination thereof. Further the server may be a cloud server system that is made up of multiple distributed hardware components. In some embodiments, sensor platforms communicate with different servers (or personal computing devices), which later can share recorded data with a central server. As can be readily appreciated, any number of different architectures can be used as appropriate to the requirements of specific applications of embodiments of the invention.

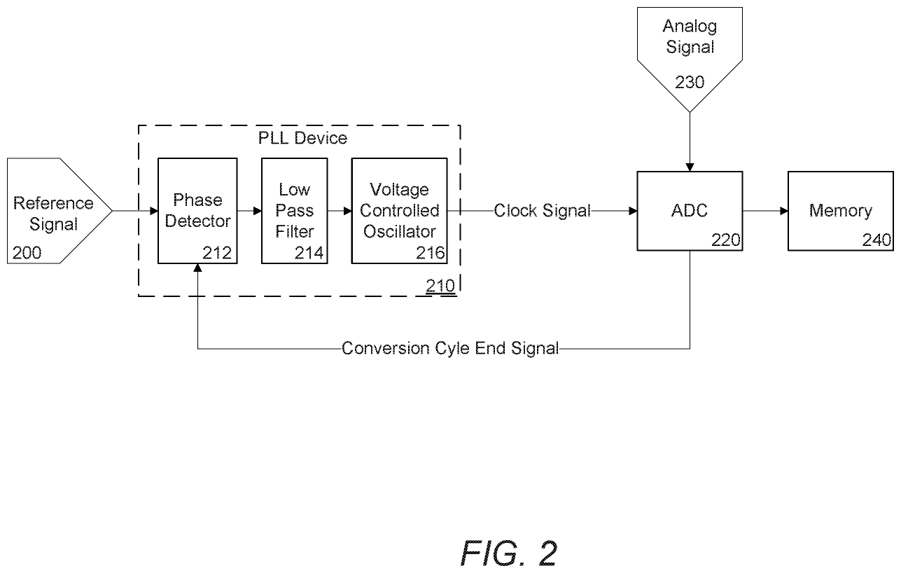

A block diagram for a sensor platform in accordance with an embodiment of the invention is illustrated in FIG. 2. In order to achieve synchronization in recording an input analog signal, a reference signal 200 is captured and input to a phase detector 212 of a phase locked loop device 210. Phase detectors are circuits which compare the phases of two signals and generate a voltage according to phase difference. Typically, in PLLs the output signal of the PLL is fed back into the phase detector in order to achieve phase locking. In the illustrated embodiment, instead of the output signal, the second input to the phase detector is a signal from an ADC 230 (e.g. a delta-sigma ADC) indicating the end of a conversion cycle. The output of the phase detector is fed into a low pass filter 214, and subsequently into a voltage controlled oscillator 216 which produces an output signal based on the input voltage. The output signal of the PLL is provided to the ADC 220 as a clock signal that controls the conversion cycle of the ADC. The analog signal 230 is then sampled over a conversion cycle window in the ADC 230, and the digital output is stored in a memory 240. However, as can be readily appreciated, the output of the ADC does not necessarily need to be immediately, or ever, stored in a memory as appropriate to the requirements of specific applications of embodiments of the invention.

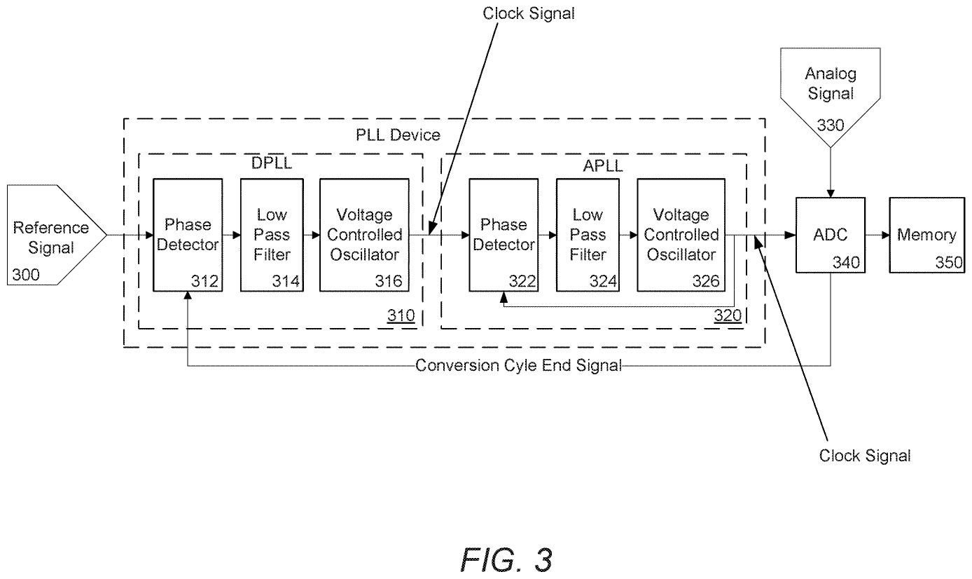

For some applications, having a cascading PLL architecture is desirable. Cascading PLLs, having both a DPLL and an APLL, can be beneficial for producing a MHz+scale clock signal from a Hz scale reference signal. In some cases, having a DPLL tune an APLL can reduce drift when the reference signal is lost. FIG. 3 illustrates a block diagram for a sensor platform having a cascading PLL architecture in accordance with an embodiment of the invention. The reference signal 300 is input into a DPLL 310, where the reference signal is first provided to phase detector 312. The second input to the phase detector 312 is the conversion cycle signal from ADC 340. In many embodiments, conversion cycle signals are signals generated by the ADC at a fixed point in a conversion cycle. For example, in numerous embodiments, the conversion cycle signal is a conversion cycle “end” signal, or a conversion cycle “start” signal. As can be readily appreciated, the point during the conversion cycle can be arbitrarily selected so long as it is repeated. The output of the phase detector is provided to a low pass filter 314, which is set based on the frequency of the reference signal. For example, if the reference signal is a 1 pps, or other low-n npss signal, the low pass filter cutoff frequency is approximately 50 mHz. The output of the low pass filter is provided to voltage controlled oscillator 316, which in turn outputs the DPLL output signal.

The DPLL output is provided to a phase detector 322 of an APLL 320, along with the output signal of the APLL. The output from phase detector 322 is provided to a low pass filter 324 which is tuned to the needs of the particular application. For example, in EEG recording, the low pass filter 324 has a cutoff frequency of approximately 100 kHz. The filtered signal is then provided to voltage controlled oscillator 326 which outputs the APLL output signal at a much higher frequency than the reference signal. The APLL output signal is provided to ADC 340 as a clock signal, and analog signal 330 is provided to the ADC for conversion and storage in memory 350. Similarly, the memory need not be incorporated, or other digital signal processing operations can take place post conversion as appropriate to the requirements of specific applications of embodiments of the invention. In many embodiments, the ADC output is timestamped. In various embodiments, the reference signal is used to timestamp the ADC output. In some embodiments, the ADC timestamps the signal based on the input clock signal. However, any number of different timestamping techniques can be used as appropriate to the requirements of specific applications of embodiments of the invention.

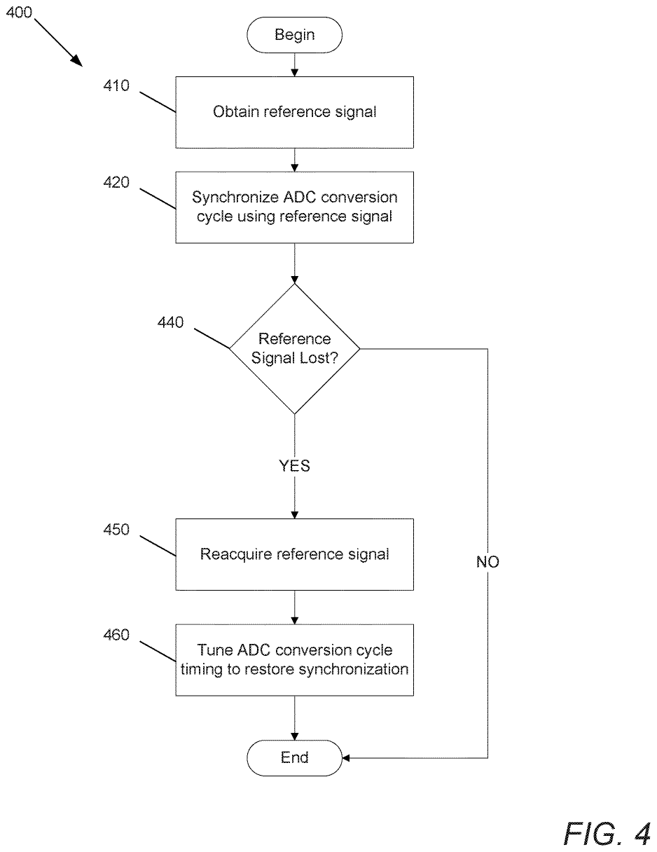

Turning now to FIG. 4, a process for continuously synchronizing an ADC in accordance with an embodiment of the invention is illustrated. Process 400 includes obtaining (410) a reference signal. The reference signal is used to synchronize (420) the ADC conversion window using a PLL and circuitry described above. If the reference signal is lost (430), the conversion by the ADC continues until the reference signal is reacquired (440), at which point the reference signal is used to tune the conversion cycle timing to restore synchronization.

While FIG. 4 illustrates synchronization of an ADC, many different devices can be synchronized using architectures and methods described herein. For example, global shutter readouts from an image sensor can be synchronized at very high frame rates by providing a “transfer complete” signal (similar to the conversion cycle signal above) rather than a conversion cycle signal. This enables highly synchronous frames captured using a very spatially distributed imaging system.

More generally, any sampling process that occurs over a window can achieve very precise global synchronization across distributed platforms across the planet so long as a reliable reference signal can be provided (e.g. a GPS signal). This is particularly useful because many sampling algorithms assume constant sampling (or frame) rates. If the assumption doesn't hold true, data is wasted as epochs (or frames) are discarded. Aside from being wasteful, this can result in short holes in time series data. Many algorithms similarly are not robust to missing data from 1 or more nodes. As the number of sensor platforms increases, missing data becomes inevitable. Systems and methods described herein address this limitation by preventing dropped epochs.

Although specifics are discussed above, many different synchronization methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A sensor platform for a distributed sensor system, comprising:
a receiver;
a phase locked loop (PLL) device, comprising:
a first phase detector configured to receive a reference signal from the receiver;
a first low pass filter configured to receive an output of the first phase detector; and
a first voltage controlled oscillator configured to receive an output of the first low pass filter; and
an analog-to-digital converter (ADC) configured to produce a digital output signal representing a plurality of samples of an input analog signal taken over a conversion cycle, where the conversion cycle timing is controlled by an output clock signal produced by the PLL device;
where the first phase detector is further configured to receive a conversion cycle signal produced by the ADC when a given conversion cycle ends.

2. The distributed sensor platform of claim 1, wherein the reference signal is a 1 pulse per second signal, and the first low pass filter cutoff frequency is between 40 mHz and 60 mHz.

3. The distributed sensor platform of claim 1, wherein the phase locked loop device further comprises:
a second phase detector configured to receive a first clock signal from the first voltage controlled oscillator and a second clock signal from a second voltage controlled oscillator;
a second low pass filter configured to receive an output of the second phase detector; and
the second voltage controlled oscillator configured to receive an output of the second low pass filter, and output a second clock signal;
wherein the second clock signal is the output clock signal.

4. The distributed sensor platform of claim 3, wherein the reference signal is a 1 pulse per second signal, and the second low pass filter has a cutoff frequency between 90 kHz and 110 kHz.

5. The distributed sensor platform of claim 3, wherein the first phase detector, the first low pass filter, and the first voltage controlled oscillator comprise a digital PLL.

6. The distributed sensor platform of claim 3, wherein the second phase detector, the second low pass filter, and the second voltage controlled oscillator comprise an analog PLL.

7. The distributed sensor platform of claim 1, further comprising a memory configured to store the digital output signal.

8. The distributed sensor platform of claim 1, wherein the ADC is a delta sigma ADC.

9. The distributed sensor platform of claim 1, wherein the input analog signal is an electroencephalography signal.

10. The distributed sensor platform of claim 1, wherein the reference signal is a global positioning system signal.

11. The distributed sensor platform of claim 1, wherein the reference signal is an n-pulse per second signal.

12. The distributed sensor platform of claim 1, the reference signal is additionally used to timestamp the digital output signal.

13. A distributed sensor system, comprising:
a plurality of distributed sensor platforms, each comprising:
a receiver;
a phase locked loop (PLL) device, comprising:
a first phase detector configured to receive a reference signal from the receiver;
a first low pass filter configured to receive an output of the first phase detector; and
a first voltage controlled oscillator configured to receive an output of the first low pass filter; and
an analog-to-digital converter (ADC) configured to produce a digital output signal representing a plurality of samples of an input analog signal taken over a conversion cycle, where the conversion cycle timing is controlled by an output clock signal produced by the PLL device;
where the first phase detector is further configured to receive a conversion cycle signal produced by the ADC when a given conversion cycle ends.

14. The distributed sensor system of claim 13, where each distributed sensor platform in the plurality of distributed sensor platforms further comprises:
a second phase detector configured to receive a first clock signal from the first voltage controlled oscillator and a second clock signal from a second voltage controlled oscillator;
a second low pass filter configured to receive an output of the second phase detector; and
the second voltage controlled oscillator configured to receive an output of the second low pass filter, and output a second clock signal;
wherein the second clock signal is the output clock signal.

15. The distributed sensor system of claim 13, wherein each ADC is a delta sigma ADC.

16. The distributed sensor system of claim 13, wherein the input analog signal is an electroencephalography signal.

17. The distributed sensor system of claim 13, wherein the reference signal is an n-pulse per second signal.

18. The distributed sensor system of claim 13, the reference signal is additionally used to timestamp the digital output signal.

19. The distributed sensor system of claim 13, further comprising a server configured to receive digital output signals from each distributed sensor platform.

20. The distributed sensor system of claim 13, further comprising a reference signal generator.
